(19) United States
(12) Patent Application Publication
(10) Pub. No.: US 2002/0127658 A1
(43) Pub. Date: Sep. 12, 2002

(54) Circularized recombinant nucleic acids and process for constructing them by means of DNA compacting agents

(76) Inventor: Michele Amouyal, Paris (FR)

Correspondence Address: Schnader Harrison Segal & Lewis, IP Department, 36th Floor, 1600 Market Street, Philadelphia, PA, 19103, US

(21) Appl. No.: 09/898,292

(22) Filed: Jul. 3, 2001

Related U.S. Application Data
(63) Continuation of application No. PCT/FR00/0004, filed on Jan. 4, 2000.

(30) Foreign Application Priority Data
Jan. 8, 1999 (FR) 99/00148

(51) Int. Cl. C12P 19/34; C12N 15/00

(52) U.S. Cl. 435/91.2; 435/320.1; 435/235.1

(57) ABSTRACT

A process for preparing circularized recombinant nucleic acids of moderate to large size by ligating an insert and a vector in the presence of a DNA compaction agent followed by selecting for the circularized recombinant nucleic acid and a kit for preparing the circularized nucleic acids in the presence of a DNA compaction agent.

The present invention pertains to the construction of circularized recombinant nucleic acids of the type constituted of a vector and an insert that can be of a size greater than several kilobases.

The invention also pertains to the process for preparation of such constructions and the kits for their implementation as well as the recombinant nucleic acids that can be obtained by this process.

The construction of DNA vectors intended to be transferred into prokaryote or eukaryote cells envisages all in-vitro and in-vivo uses of these sequences such as analysis of the biological effects of the DNA sequence (effect of the DNA or of its expression), expression of RNA, expression of proteins, amplification of hybridization probes for medical diagnosis, cell or gene therapy, vaccination, etc.

Recombinant vector construction, comprising insertion of a DNA fragment in a vector, includes an in-vitro vector reclosure step. In order for reclosure to take place, the ends of the finished vector must be close to each other. These ends move within a sphere with a radius equal to the length of the fragment and with one of the ends as its center. The greater the size of the finished vector, the greater the volume of this sphere. Consequently, the probability of conjunction of the two ends decreases with the length of the finished vector.

With regard to insertion of the fragment in the vector, it takes place if the probability of conjunction between the ends of the insert and those of the original vector is high. Thus, insertion is dependent on the concentration of ends.

In other words, the proportion of inserted and cyclized products is dependent on the length of the finished vector for the intramolecular reclosure process and on the total concentration in free ends for the intermolecular ligation process.

The very first generation vectors are vectors of bacterial origin derived from plasmids. Other vectors take into account more precisely the specificities of the eukaryote genes, in particular their size. This is the case of the cosmids, hybrid compounds of phage 1 and plasmids reproducing in E. coli and the YACs whose host organism is yeast.

The cosmids enable insertion up to 45 kb, the YACs up to 1000 kb. These vectors are intended for analysis of genomic DNA libraries or chromosomal analysis. Their efficacy is linked to the range of different fragments that they can receive. Their use is burdensome if it is a question of only inserting a specific fragment and amplifying it. Thus, in order to preserve their stability, the insertion vector must conserve a minimum size (33 kb for the cosmids, 150 kb for the YACs). However, handling such lengths of DNA is fraught with numerous problems (breaks, cuts, etc.), whereas the sequence under consideration is frequently of smaller size, only reaching several kb when complementary DNA is preferred to genomic DNA. Moreover, replication in E. Coli of vectors of large size such as cosmids as well as the stability of YACs in yeast are limited, which leads to sequence modifications. In the case of the cosmids, the efficacy of the DNA packaging and the infection by the phage 1 compensates for this disadvantage to a certain extent.

Another insertion technique comprises the use of adjuvants of a chemical nature such as polyethylene glycol (Zimmerman S. B. and Pheiffer B. H., Proc. Natl. Acad. Sci. USA, 80, 5852 (1983) or hexamine cobalt chloride (Maniatis et al., Molecular Cloning / A Laboratory Manual, second edition, 1989) for straight-end ligations. These ligations are not as easy to implement as cohesive-end ligations, and require higher concentrations of DNA and ligase. The purpose of these chemical adjuvants is to promote aggregation between insert and vector (intermolecular reaction) and to decrease the concentrations of DNA and ligase; they do not apply to the construction of vectors of large size.

At present, there is no technique which facilitates the circularization and preparation of vectors which is effective for moderate sizes, several thousand of base pairs, as well as for larger sizes, above 10 kb.

The term vector of large size is understood to means a vector of at least 10 kb in which is integrated an insert of multiple kilobases.

Cosmids and YACs are responsive to the analysis requirements and search for as broad as possible an insertion range from DNA libraries. The presently employed ligation adjuvants are never proteins naturally designed to condense the DNA and do not promote the insertion of fragments of large size or reclosure of the recombinant.

The goal of the present invention is to facilitate the circularization and preparation of vectors, more particularly vectors of large size.

In the method according to the present invention, it is the presence of compaction protein during ligation which increases the level of ligation products and the probability of amplifying the correct product.

In fact, one of the values of the invention is in the domain of cloning which generally includes a prior step of in-vitro cyclization of the recombinant. The efficacy of the cyclization depends on the size of the recombinant and is improved by compaction of the DNA. In fact, vector construction includes an in-vitro vector closure step. The efficacy of this closure depends on the size of the DNA fragment.

Consequently, the object of the present invention is a process for preparation of circularized recombinant nucleic acids of the type constituted of a vector and an insert, characterized in that:

a) ligation of the insert and the vector is implemented in the presence of a DNA compaction agent, and

b) the constituted recombinant nucleic acids of the vector and the insert are selected by any suitable means.

Thus, one application of the process of the invention is the cloning of an insert in a vector under the conditions defined above, enabling advantageously production of a recombinant vector of large size.

The process of the invention is especially suitable for preparation of circularized recombinant nucleic acids of moderate or large size. In other words, of a size greater than several kb, preferably greater than 5 kb, and especially preferably greater than 8 or 10 kb.

The phrase “by any suitable means” in step (b) should be understood to mean the transfer of the ligation products into a cellular medium suitable for DNA cloning, e.g., E. coli or yeast, in the presence of an antibiotic such as ampicillin or tetracycline, if the vector carries an antibiotic resistance gene, and a test for the presence of the insert, for example, in the case of the control gene lacZ, hydrolysis of the x-gal compound by the b-galactosidase produced.

However, all other selection means can be used, particularly those suitable for the production of DNA to pharmaceutical standards for gene or cell therapy. Similarly, the step does not necessarily include a specific test for the presence of the insert since insertion can be verified by DNA sequencing after production of the vectors by the process of the invention.

Histones are the most abundant proteins in the nucleus; they are of small size (11 to 25 kDa) and of a very basic nature (pH>10). There are five types of histones which are referred to as H1, H2A, H2B, H3 and H4, respectively. These five types are found with variants in all of the eukaryotes (with the exception of H1 which does not appear to exist in yeast, and which is replaced by histone H5 in certain organisms). H2A, H2B, H3 and H4 are found in vivo in octamer form. The DNA coils twice around the octameric core so as to form a nucleosomic structure. The histone H1 does not participate in this nucleus but serves to seal the DNA around the octamer. In the eukaryote cell, the compaction of the DNA by the histones has the effect, notably, of bringing close to each other two transcriptional regulation sites which are situated remotely from each other on the chromosome, and to enable formation of a chromatin loop by direct interaction between these sites. Thus, the expression of a gene can be controlled remotely by these genes (Amouyal, Biochimie (1991), 73, p. 1261-1268).

According to the invention, ligation of the insert and of the original vector is implemented in the presence of DNA compacting products. The insertion vector contains a replication origin and, advantageously, a selection gene for growth and selection in the cell type under consideration.

The DNA compaction agents are proteins, mixtures of proteins or protein derivatives. The term “proteins” is employed to indicate natural or synthetic proteins.

The DNA compacting products are proteins or any agents exhibiting the same properties, and more particularly the histone proteins or related proteins. In addition to the histones, the DNA compaction agent can be selected from all the proteins known to compact DNA, especially the viral or phage envelope proteins, the bacterial chromoid proteins (HU, H-NS, etc.), the non-histone chromosomal proteins, the HMGs, etc., all mixtures of these compounds, or any derivative thereof.

Therefore, for step (a) of the process according to the invention, the DNA compaction agent is selected from among the histones, the viral or phage envelope proteins, the bacterial chromoid proteins (HU, H-NS, etc.), the non-histone chromosomal proteins, the HMGs, any mixture of these compounds or any derivative thereof.

Or a mixture of said condensation agents.

The DNA compaction agent is preferably a mixture of histones.

The inventor has demonstrated that a mixture of histones or an isolated histone lead to similar results. The histone H1, of different structure and not being a histone forming the octamer but rather a sealing histone, used by itself does not appear to yield results as good as the other histones.

This is explained by the fact that, because of its different structure, H1 does not bind to linear DNA but rather preferably to supercoiled DNA (Van Holde et al., Biophysical Journal, 1997, 72, p. 1388-1395).

In a preferred mode of implementation of the invention, the compaction agents are added to the ligation medium. This medium comprises DNA in solution in the ligation buffer. Ligation takes place by addition of a ligation enzyme to the ligation medium.

In a preferred but nonlimitative manner, the ligase employed is E. Coli T4 DNA ligase.

One characteristic of the invention is based on the concentration (C) of compaction agents present in the ligation medium.

This compaction agent concentration (C) is determined so as to not cause a rigidification of the DNA. If rigidification takes place, the DNA can not bend, there will not be any contact between the ends of the finished vector and ligation will be impeded.

For a given protein or mixture of proteins, the amount of compaction agent is defined by calculation and tested by gel retardation assay, so as to not saturate a DNA-control fragment in proteins.

This concentration (C) can be expressed in mg of proteins per nanogram of total DNA contained in the ligation mixture and by base pair of recombinant. The concentration is dependent on the length of DNA to be ligated and the inventor has defined a multiplicity of laws, each corresponding to the protein agents employed:

Thus for the natural mixture of histones Sigma,

(C)=1.5·10⁻¹¹ mg/ng DNA/bp

for the histone H2B Sigma,

(C)=1.5·10⁻¹² mg/ng DNA/bp.

By extrapolation, the inventor defined the following law which can be applied to all of the compacting proteins employed:

(C)=10^{31 x} mg/ng DNA/bp

in which x is between 8 and 15 inclusively.

The value x is a function of the nature of the compaction agent employed.

In practice, the efficacy of the cloning is always improved if the concentration is within a range encompassing the value thereby defined and extending from 20 to 1000%, and preferably between 33 and 200% of this value.

The process according to the present invention is thus characterized in that the concentration(C) of compaction agent is determined by the following law:

(C)=(10^{-x} mg/ng DNA/bp) ×Y

in which:

×is comprised between 8 and 15, preferably between 10 and 12, and

Y varies between 0.2 and 10, preferably between 0.33 and 2.

The present invention also pertains to a kit for the implementation of the method for the preparation of circularized recombinant nucleic acids of large size as defined above. This kit comprises the following reagents:

a ligation buffer,

a ligase,

a compaction agent as defined above.

As preferred compaction agent, a mixture of histones or an isolated histone would be used.

Another characteristic of the invention pertains to the parameters for implementation of the method and the kit comprising the object of the invention. Ligation is implemented under the usual conditions with the modifications specified below.

The usual conditions are dependent on the ligase employed and the indications specified by the company marketing the enzyme. For T4 DNA ligase marketed by NEB Biolabs, the ligase is employed with a buffer:

5 mM Tris HCl

1 mM MgCl₂

1 mM DTT

0.1 mM ATP

2.5 μg/ml BSA.

Prior to ligation, the protein agent is put into solution in the ligation buffer—possibly containing glycerol—(or diluted in the ligation medium, if the agent is provided in solution form). It is incorporated in the ligation medium in the proportions defined by the law specified above.

In a preferred manner, the kit includes a stabilizing agent incorporated in the ligation medium at the same time as the protein, which agent is designed to prevent the denaturation, aggregation and/or adsorption of the protein on the walls of the reaction tube at these strong dilutions. Thus, a kit according to the invention comprises:

a ligation buffer,

a ligase,

a compaction agent,

possibly a stabilizing agent for the protein.

In a preferred manner, this dilution buffer is glycerol or any compound presenting the same characteristics.

A preferred kit according to the invention comprises:

the ligase is E. coli T4 ligase,

the corresponding ligation buffer,

one or more histones as compaction agent,

the stabilizing agent, if present, is glycerol.

The invention also pertains to the recombinants, preferably of large size, which can be obtained by means of the process described above.

Such a recombinant can be constituted by a vector containing a replication origin, possibly a selection gene, the insert, possibly associated with an indicator gene enabling detection of the presence of the insert such as the b-galactosidase gene or a selection gene.

The applications of the invention are, of course, to be found in the field of cloning, but also in the domain of gene therapy, most especially in the context of genetic vaccination.

Thus, the invention pertains more specifically to a circularized recombinant nucleic acid of large size greater than several kilobases constituted by a vector and an insert. In a first form of implementation of the invention intended for gene therapy, said insert comprises one or more cDNA coding for one or more proteins required for the correction of a genetic deficiency, placed under the control of sequences enabling their in-vivo expression.

In a second form of implementation of the invention intended for genetic vaccination, said insert comprises one or more DNA coding for one or more antigens, placed under the control of sequences enabling their in-vivo expression. More specifically, said insert comprises one or more, and preferably the totality, of the DNA sequences coding for antigens capable of inducing an immune reaction.

According to one preferred implementation, the vector is a nonviral vector, albeit capable of containing viral elements.

Other advantages and characteristics of the invention will be perceived from the examples below which are presented as nonlimitative examples, and pertain to the construction of a recombinant of large size by means of DNA compacting proteins, and to the cloning of said recombinant in E. coli, presented with reference to the attached drawings in which:

FIG. 1 shows the complexation of the protein to the fragment fR4 under ligation conditions followed by retardation of the electrophoretic migration of the fragment in 0.4% agarose gel.

1a: Trial 1

1b: Trial 4

1c: Trial 1 with purified protein H1 (example 2)

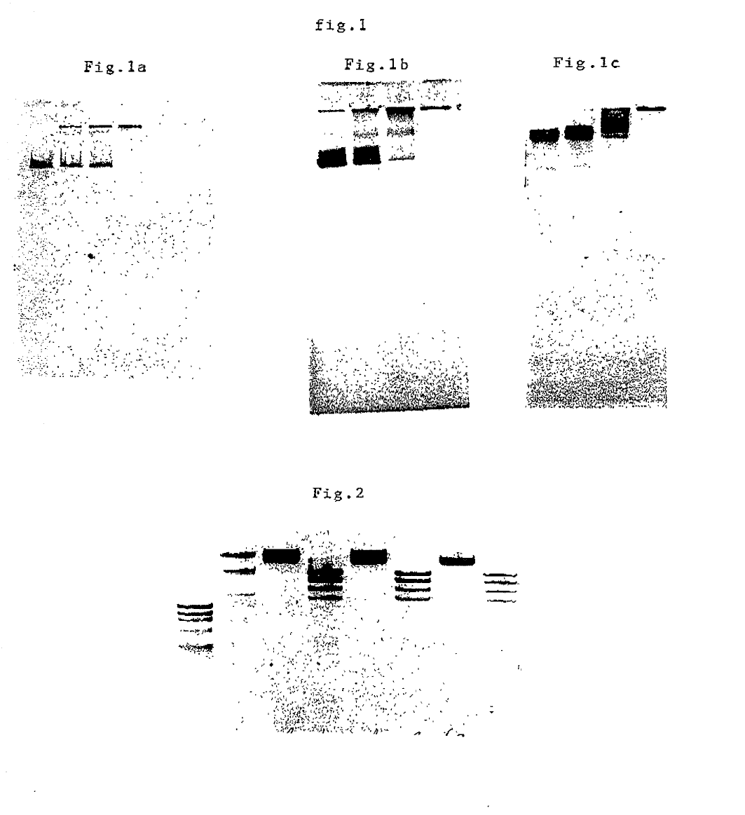

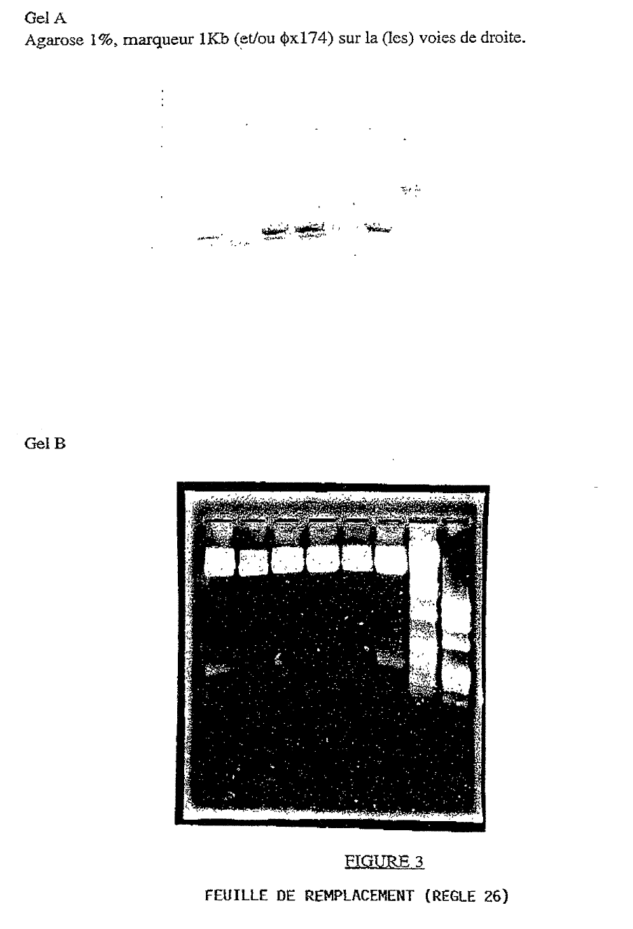

FIG. 2 shows the digestion by EcoRI of the three recombinants R4-LZ with insert obtained in trial 11.

FIG. 3 shows the digestion by EcoRI of 6 of the 20 recombinants K-LZ (example 1b).

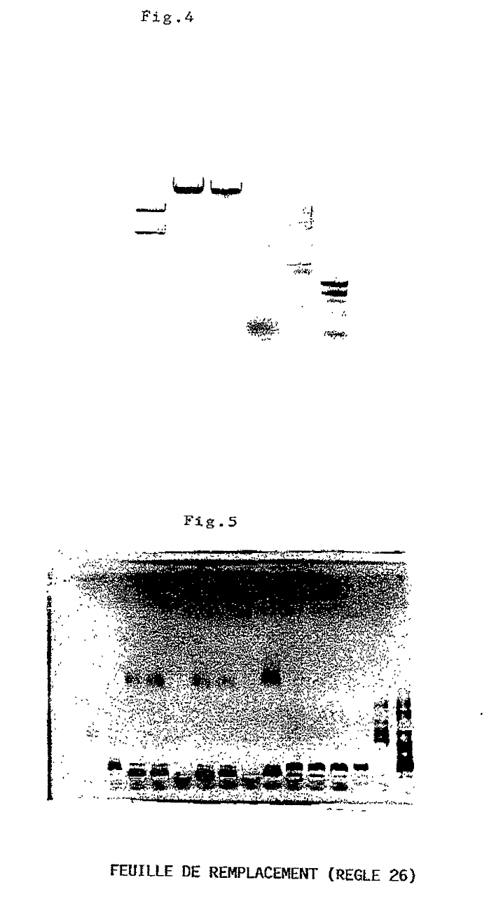

FIG. 4 shows the augmentation of the quantity of ligation products in the presence of histones, 0.4% agarose gel.

FIG. 5 shows the sensitivity test to the nucleases possibly present in the histone preparation.

### EXAMPLE 1

### LIGATION IN THE PRESENCE OF A MIXTURE OF HISTONES, SELECTION AND AMPLIFICATION OF THE LIGATION PRODUCTS BY E. COLI

Two recombinants of different sizes were constructed. The first (recombinant R4-LZ) has a size of 12,034 base pairs; the second and smaller (recombinant K-LZ) has a size of 6,785 bp.

### Example 1a

### Cloning a Recombinant of 12,034 bp

I-Material and Methods

I.1. Fragments

This first recombinant R4-LZ of a size of 12,034 base pairs stems from the ligation of two fragments fR4 and fLZ,

Fragment of 8,159 base pairs: fR4

This fragment stems from the plasmid pREP4 (Invitrogen) by complete digestion of the 10,183 -bp plasmid by the restriction enzymes SalI (positions 7 and 1091) and SpeI (unique site at 9,250).

The fragments obtained by digestion have the respective sizes of 8,159, 1,084 and 940 bp.

The 8,159-bp fragment contains the replication origin colE1 and the ampicillin-resistance gene of E. coli. It is separated from the other fragments by electrophoretic migration on low-melting-point agarose gel (Seaplaque LMP agarose, FMC Bioproducts), excision and extraction of the corresponding band.

Fragment of 3,875 base pairs: fLZ

This fragment stems from a 6,206-bp plasmid containing the gene lacZ under the control of a promoter of E. coli (construction derived from the plasmid pUT79 from the Cayla company, Toulouse).

By digestion at the unique sites SpeI (position 29) and SalI (position 3904) followed by electrophoretic separation, the 3,875-bp fragment containing the gene lacZ under control of the bacterial promoter was separated from the 2,331 -bp fragment.

I.2. Compacting protein

This is the type IIA preparation (reference H9250 Sigma) containing all of the calf serum histones without fractionation.

This preparation, which is marketed in lyophilized form, is put in solution in the ligation buffer just before use in the previously specified concentrations.

I.3. Ligations

The two fragments fR4 and fLZ were mixed in the previously specified proportions in 20 μl of ligation buffer for T4 DNA ligase of E. coli. These ligation conditions are close to those employed conventionally (see Current Protocols and Maniatis).

When necessary, the protein is added to the mixture of fragments at the desired concentration. The T4 DNA ligase is added after incubation in a period of time between 0 to 20 minutes, more particularly between 3 and 5 minutes.

1.4. Cellular transformation by the ligation medium

The strain DH5a is a strain deficient in protein RecAl, which does not promote recombinations and rearrangements of the DNA within the cell (notably, the plasmids remain in monomer form while deletions are avoided, pages 4-13 of Maniatis).

The strain is transformed by 10 μl of the ligation mixture according to a variant of Hanahan's method (J. Mol. Biol. 166, 557, 1983) leading to an efficacy of 10⁷ colonies/μg of DNA. Selection of the cells containing the recombinant is performed on LB medium with a concentration of 200 μg/ml of ampicillin, and Petri dishes covered with 200 μl of X-gal in solution in DMF.

1.5. Histone concentration

A first histone concentration value was determined by calculating the quantity of the natural mixture of histones assumed to be necessary for the formation of one nucleosome every 200 base pairs, and choosing deliberately to only take a part of the amount of protein thereby determined (one fourth in this example) so as to avoid saturation of the fragment in protein. The law derived from this is the following:

(C)=1.2·10⁻¹¹ mg of histone mixture/ng of DNA/bp of recombinant

This law was applied to the complexation of the longer fragment, i.e., fR4 in the present case. Complexation was monitored by gel retardation assay in a 0.4% agarose gel in the presence of the determined concentration and two other flanking quantities at 50% and 200% (FIG. 1).

The law, which was readjusted on the basis of these gels and which was subsequently employed, is very close to the calculated value:

(C)=1.5·10⁻¹¹ mg of the histone mixture/ng of DNA/bp of recombinant

Each ligation was performed in the presence of three concentrations of proteins, the quantity derived from this law two and quantities encompassing this quantity and corresponding to 50 and 200% of the calculated quantity, respectively.

On these gels, we have:

Track 1: Fragment fR4 without proteins (except in the gel corresponding to trial 4),

Track 2: Fragment fR4+0.025 μg of protein,

Track 3: Fragment fR4+0.05 μg of protein,

Track 4: Fragment fR4+0.1 82 g of protein,

Track 5: Fragment fR4+0.2 μg of protein (trial 4).

For 500 ng of fragment fR4, 0.025 μg, 0.05 μg and 0.1 μg of the histone mixture satisfy the conditions.

The ligations take place at the three corresponding concentrations.

1.6. Verification of the absence of sequence rearrangements

The first tests to verify production of a recombinant whose sequence did not undergo rearrangements is the resistance to ampicillin conferred on the cell and its capacity to express b-galactosidase (blue colonies in the presence of X-gal).

Supplementary verification is implemented by enzymatic digestion. Thus, cleavage by EcoRI of a correctly bound and reclosed recombinant R4-LZ should result in 6 fragments of the respective sizes: 4,172, 3,076, 2,875, 2,021, 393 and 283 bp.

These results are illustrated by FIG. 2 in which:

Track 1: marker fₓ 174/HaeIII

Track 2: 1-Kb marker

Tracks 3, 5, 7: recombinants without cleavage

Tracks 4, 6, 8: digestion of these same recombinants by EcoRI

1.7. Other preliminary tests

The transformations of E. coli were implemented with the ligation mixture containing only one of these two fragments, in the presence of or absence of histones, to confirm the electrophoretic purity of the fragments and the absence of reclosure within the cell. A gel retardation assay was performed on the fragment fR4 prior to each ligation with this fragment in order to monitor the complexation of the DNA by the protein. Separation was performed on standard 0.4% agarose gel. This gel assay was performed under the same conditions as the ligation experiments in 20 μl deposited on gel and is shown in FIG. 4, in which:

Track 1: ligation mixture (recombinant R4-LZ) without histones (fragments fR4-8159 bp and fLZ-3875 bp) arrows

Track 2: ligation mixture in the presence of 0.025 μg of histones

Track 3: ligation mixture in the presence of 0.05 μg of histones

Track 4: purified recombinant R4-LZ

Track 5: 1-kb marker (Gibco BRL)

Track 6: marker f₃₃ 174/HaeIII (Gibco BRL)

II-Results

The first 13 trials were performed on the basis of the quantity of protein determined by gel retardation assay from the complexation of the fragment fR4 by itself rather than the law stemming from these experiments. However, the size (8159 bp) of the fragment fR4 represents only ⅔ of the total size of the recombinant. By employing this values for the ligations, the law was only followed by two thirds (⅔ law). The more recent trials, which were also the more successful, were performed by taking into account the quantity of protein (⅓ more) that needed to be added to complex the entire vector and insert unit in the same manner as the fragment by itself.

The presence of the lacZ inserts was tested by the capacity of the selected colony to hydrolyze the X-gal compound (blue coloration) and by the production of a correct profile of enzymatic cleavage (EcoRI test) as specified in the legend to FIG. 2.

The number of R4-LZ recombinants (12,034 bp) obtained from the ligations in the presence of the histone mixture is presented below:

| Trial | 1 | 2 | 3 | 4 | 4′ | 5 | 6 |
|---|---|---|---|---|---|---|---|
| (− protein) | 0 | 0 | 0 | 0 | | 0 | 0 |
| (+ protein) | | | | | | | |
| Total number | 3, 0, 0 | 0, 0, 1 | 2, 0, 0 | 1, 4, 0, 0 | 7, 3, 0, 0 | 0, 0, 1 | 0, 0, 0 |
| With lacZ insert | 3, 0, 0 | 0, 0, 1 | 0, 0, 0 | 0, 0, 0, 0 | 0, 2, 0, 0 | 0, 0, 1 | 0, 0, 0 |

| Trial | 7 | 8 | 9 | 10 | 11 | 11′ | 12 |
|---|---|---|---|---|---|---|---|
| (− protein) | 0 | 0 | 0 | 0 | 0 | 0 | 1 (without |
| | | | | | | | insert) |
| (+ protein) | | | | | | | |
| Total number | 0, 0, 0 | 0, 0, 0 | 0, 1, 0 | 3, 5, 3 | 0, 3, 2 | 1, 1, 0 | 1, 2, 2 |
| With insert | 0, 0, 0 | 0, 0, 0 | 0, 1, 0 | 1, 1, 0 | 0, 3, 2 | 0, 0, 0 | 0, 2, 1 |

| Trial | 12′ | 12″ | 12″′ | 13 | 14 | 15 |
|---|---|---|---|---|---|---|
| (− protein) | | | | 0 | 0 | 0 |
| (+ protein) | | | | | | |
| Total number | 1, 2, 2 | 4, 3, 3 | 1, 3, 1 | 0, 0, 0 | 11, 8, 2 | 0, 1, 0 |
| With insert | 1, 0, 0 | 3, 2, 1 | 0, 2, 1 | | 9, 2, 2 | 0, 1, 0 |

II.1 Trials 1-6, 9-13: ⅔ law

Trials 4 and 4 ′: 4 quantities of protein were tested: 50, 100, 200, 400%

Trial 7: quantities of protein 3 times higher (150, 300, 600%)

Trial 8: quantities of protein 10 times higher (500, 1000, 2000%) (to compensate for a possible complexation deficit due to a lower concentration of DNA

Trials 14 and 15: exact law

II.2. Effect of storage and freezing of the protein:

Trial 2: dilution of the protein from a stock-solution of 100 mg/ml in the dilution buffer containing 50% glycerol, stored at −20° C. to test the effect of storage. For all of the other trials, the protein solution was freshly prepared from the lyophilized powder.

II.3. Quantity of DNA

Trials 1-5, 9-14: fragment fR4: 370 ng fragment fLZ: 190 ng (equimolecular quantities of vector and insert, 10 μl of ligation medium)

Trials 6, 7 and 15: fragment fR4: 150 ng fragment fLZ: 75 ng (2.5 times weaker equimolecular quantities, 10 μl)

Trial 8: fragment fR4: 150 ng fragment fLZ: 190 ng (3 times more of fLZ molecules than fR4 molecules, 10 μl)

II.4. Order of addition of certain components to the ligation medium

Trial 3: the fLZ fragment was added at the same time as the ligase after incubation of fR4 with the histones as in the gel retardation assay. This process was subsequently avoided because it probably promotes reclosure of the vector without insert.

II.5. Effect of freezing the ligation mixture

Trials 4 and 4′:

Trial 4 was implemented with 20 μl of ligation mixture. After addition of the ligase, half of the mixture was frozen at −20° C. for a subsequent transformation (trial 4′). Freezing does not appear to have a negative effect on the efficacy of the histones.

II.6. Presence of glycerol in the dilution buffer

Trials 9, 10, 11′, 12′, 12′″, 14, 15

Glycerol is usually added to the storage buffer, as above, to prevent denaturation and aggregation of the proteins during the successive freezing-thawing cycles. Thus, 0.1% of glycerol was added to the protein dilution buffer (ligation buffer) so as to prevent aggregation or denaturation of the protein when it was put into solution, as well as to avoid the loss of protein on the tube walls in the final dilutions. In trial 11′, glycerol was added on a delayed basis (after the first ligation series of trial 11, i.e., after approximately ten minutes).

II.7. Incubation time with the histones

Trials 11′, 12′, 12′″:

15 minutes rather than a maximum of 5 minutes

We always selected a part of the recombinants not possessing the anticipated sequence by cloning in E. Coli, even in the absence of protein adjuvants. These defects are generated at the time of in vitro ligation or by incorrect replication of the DNA by the cell.

In this example, the presence of incorrect recombinants is demonstrated by the presence of white colonies.

In order to determine whether the presence of these incorrect recombinants is due specifically to the use of a protein preparation, especially to the possible presence of nucleases, and to the partial degradation of the DNA which could result from it, a DNA sensitivity test to the nucleases in the histone preparation employed was performed. For this test, the fragment fR4 or the plasmid pR4 was incubated with the quantities of protein indicated on tracks 3 to 10. The results are illustrated in FIG. 5, in which:

Track 3: fragment fR4 (500 ng)

Track 4: fragment fR4+0.025 μg of histones

Track 5: fragment fR4+0.05 μg of histones

Track 6: fragment fR4+0.1 μg of histones

Track 7: plasmid pR4 (500 ng)

Track 8: plasmid pR4+5 μg of histones

Track 9: plasmid pR4+0.025 μg of histones

Track 10: plasmid pR4+0.05 μg of histones

Tracks 11, 12, 13: like tracks 8, 9, 10. Incubation at 20° C. for 1.5 hours rather than 20 hours.

Tracks 1 and 14: 1-Kb marker (Gibco BRL)

Track 2 and 15: marker fₓ174/HaeIII (Gibco BRL)

No nuclease digestion was detected under these conditions.

The most successful experimental conditions (those that enabled production with certitude of at least one recombinant even if the other conditions were modified, or those conditions that introduced an improvement in the cloning efficacy) were combined when the quantities of protein were determined on the basis of the law defined above, when the protein solution was freshly prepared, the incubation time short (less than 5 minutes) and the DNA concentrations sufficient (370 ng of fragment fR4 and 190 ng of fragment fLZ for 10 μl of ligation medium for the example above).

The presence of glycerol when the protein is put into solution appears to be beneficial to the cloning efficacy in the case of the natural histone mixture. It does not appear to be especially beneficial with the isolated histones (H1 or H2B, example 2). That is why the kit can contain (irrespective of the protein employed) a dilution buffer containing glycerol (or any other agent with the same properties) as well as a dilution buffer without glycerol (which is also the ligation buffer).

Comment:

Example 1a shows both the cloning of a 3,875-bp insert in an 8159-bp vector as well as the cloning of an 8,159-bp fragment in a 3,875-bp vector.

### Example 1b

### Cloning of a 6.785-bp Fragment

I-Material and Methods

I.1. Fragments

Fragment of 2,910 base pairs: fK.

This fragment stems from the digestion of the pBlueScript KS plasmid (Stratagene) by SpeI and SalI. It contains a replication origin for E. coli and the ampicillin resistance of E. coli.

I.2. Verification of the absence of sequence rearrangements

Cleavage by EcoRIs of the recombinant K-LZ should be translated by 3 fragments of 3,426, 3,076 and 283 bp. Similarly, these results are illustrated by FIG. 3 (1% agarose, 1-Kb markers (and/or fₓ174) on the tracks at right).

II-Results

The trials were implemented with equimolecular quantities of vector fK (180 ng/ 10 μl, 2,910 bp) and insert fLZ (245 ng/10 μl, 3,875 bp) at the same concentrations in vector and insert as in the preceding example (recombinant R4-LZ).

The quantities of protein employed corresponded to 33, 67 and 134% of the quantity indicated by the law specified above (⅔ law).

The number of K-LZ recombinants (6,785 bp) obtained by ligation in the presence of a mixture of histones and cloning in E. coli is presented below:

| | Trial 1 | Trial 2 |
|---|---|---|
| (− protein) | 8 | 2 |
| (+ protein) | | |
| Total number | 4, 7, 20 | t.n., 5, 5 |
| With insert | t.n., t.n., 17 | t.n., 5, 4 |

The presence of histones at the time of ligation also improves the cloning efficacy.

### Example 1c

### Reclosure of an 8,159-bp Fragment

This is the 8,159-bp fragment fR4 that has already been described.

| | Trial 1 | Trial 2 |
|---|---|---|
| (− protein) | 0 | 0 |
| (+ protein) | 5, 1, 9 | 0, 0, 0 |

Trial 2 was performed with 150 ng of fragment fR4 rather than 370 ng for trial 1 and 10 μl of ligation medium. The protein was solubilized in the presence of glycerol in trial 1 and without glycerol in trial 2.

### EXAMPLE 2

### LIGATION IN THE PRESENCE OF ISOLATED HISTONES AND CLONING OF THE 12,034-bp RECOMBINANT FROM E. COLI

The two histones employed were calf thymus histone H1 (type III-SS, ref. H4524, Sigma) and calf thymus histone H2B (type VII-S, ref. H4255, Sigma).

The law calculated for H1 was calculated on the basis of a quantity equivalent in moles to the quantity present in the natural mixture, i.e., 6 times less (molecular weight of the mixture, M=130,268; molecular weight of the protein H1, M=21,500). This law was tested by gel retardation assay (FIG. 1c).

### Example 2a

### Ligation in the Presence of Histone H1

The number of recombinants obtained was the following:

| Trial | 1 | 2 | 3 | 4 | 5 |
|---|---|---|---|---|---|
| (− protein) | 1 (w/o insert) | 0 | 0 | 0 | 0 |
| (+ protein) | | | | | |
| Total number | 1, 2, 4 | 2, 0, 0 | 0, 0, 0 | 0, 0, 0, | 0, 0, 0 |
| With insert | 0, 1, 1 | 0, 0, 0 | 0, 0, 0 | 0, 0, 0 | 0, 0, 0 |

Trials 1, 2, 3: quantity of histone Hi equivalent in moles to the quantity of histone H1 present in the mixture (trials 1 and 2: ⅔ law; trial 3: exact law).

Trials 4 and 5: quantity of histones H1 equivalent in moles to the total quantity of histones present in the natural mixture, by replacing the other histones

Trial 5: quantity of DNA 5 times less than in the other trials

Trials 1 and 2: glycerol added to the dilution buffer of the histone H1

Trial 3: without added glycerol.

Histone H1 does not increase the cloning efficacy in as pronounced a manner as the natural histone mixture under the same conditions.

### Example 2b

### Ligation in the Presence of Histone H2B

In this case, the protein (M=13,774) represents one tenth of the mixture (M=130,268), and the law becomes:

(C)=1.5·10⁻¹² mg H2B/ng DNA/bp of recombinant

The number of recombinants obtained was the following:

| Trial | 1′ | 2′ | 3′ | 4 | 4′ |
|---|---|---|---|---|---|
| (− protein) | 1 | 0 | 0 | 5 (without | |
| | | | | insert) | |
| (+ protein) | | | | | |
| Total number | 1, 1, 2 | 5, 1, 0 | 25, 0, 0 | 26, 5, 7 | 12, 18, 9 |
| With insert | 0, 0, 0 | 2, 0, 0 | 15, 0, 0 | 13, 3, 3 | 4, 6, 2 |

Trials 4, 4′, 3′: without glycerol, exact law

Trials 1′ and 2′: with glycerol, ⅔ law

Trials 1 ′, 2 ′ and 3 ′ were performed in parallel with trials 1, 2 and 3 implemented in the presence of protein H1 (same preparation of DNA, buffer and culture dishes) and in parallel with trial 14 with the mixture of histones).

In contrast to histone H1, histone H2B by itself increases the cloning efficacy.

1. Process for preparation of circularized recombinant nucleic acids of the type constituted of a vector and an insert, characterized in that:
a) ligation of the insert and the vector is implemented in the presence of a DNA compaction agent, and
b) the constituted recombinant nucleic acids of the vector and the insert are selected.

2. Process according to claim 1, characterized in that the circularized recombinant nucleic acids present a size greater than 5 kb, and preferably superior to 8 or 10 kb.

3. Process according to either one of claims 1 or 2, characterized in that step (b) is implemented by means of the transfer of the products obtained in step (a) into a cellular medium suitable for cloning DNA.

4. Process according to any one of the preceding claims, characterized in that step (a) is implemented in the presence of a DNA compacting protein or mixture of proteins.

5. Process according to claim 4, characterized in that said proteins are selected from among the histones, the viral or phage envelope proteins, the bacterial chromoid proteins (HU, H-NS, etc.), the non-histone chromosomal proteins, the HMGs, a mixture of these compounds, or derivatives thereof.

6. Process according to any one of the preceding claims, characterized in that the concentration (C) of compaction agent does not lead to a rigidification of the DNA.

7. Process according to any one of the preceding claims, characterized in that step (a) of ligation of the insert and the vector in the presence of a DNA compaction agent is performed in a ligation medium constituted by a ligase and a corresponding buffer.

8. Process according to one of claims 1 to 7, characterized in that the ligase is E. coli T4 ligase.

9. Kit for the implementation of any one of claims 1 to 8, characterized in that it comprises:
a ligase,
a ligation buffer corresponding to the ligase,
a compaction agent,
possibly a stabilizing agent.

10. Kit according to claim 9, characterized in that:
the ligase is E. coli T4 ligase,
the corresponding ligation buffer,
the compaction agent is a mixture of histones or an isolated histone,
if present, the stabilizing agent is glycerol.
